(19) United States
(12) Patent Application Publication
(10) Pub. No.: US 2008/0275439 A1
(43) Pub. Date: Nov. 6, 2008

(54) CARDIAC ABLATION AND ELECTRICAL INTERFACE SYSTEM AND INSTRUMENT

(76) Inventors: David Francischelli, Anoka, MN (US); Mark Stewart, Lino Lakes, MN (US); Daniel Cheek, Plymouth, MN (US); Brian Ross, Maple Grove, MN (US); Paul Rothstein, Elk River, MN (US); Scott Klein, St. Paul, MN (US); Asif Hameed, St. Michael, MN (US); Doug Crowe, Blaine, MN (US); Kester Batchelor, Minneapolis, MN (US); William Colon, Minneapolis, MN (US)

Correspondence Address: DICKE, BILLIG & CZAJA, PLLC;ATTN: MD MATTERS, FIFTH STREET TOWERS, SUITE 2250, 100 SOUTH FIFTH STREET, MINNEAPOLIS, MN, 55402, US

(21) Appl. No.: 12/055,991

(22) Filed: Mar. 26, 2008

Related U.S. Application Data
(63) Continuation-in-part of application No. 10/853,594, filed on May 25, 2004.
Application No. 10/853,594 is a continuation-in-part of application No. 10/056,807, filed on Jan. 25, 2002.

(51) Int. Cl. A61B 18/12

(52) U.S. Cl. 606/34

(57) ABSTRACT

A system for ablating tissue and electrically interfacing with a heart including an electrosurgical instrument, an energy source, and a controller. The instrument includes a shaft maintaining first and second electrodes at a distal section. The electrodes are electrically isolated from one another. The controller controls delivery of energy from the energy source, and monitors electrical signals at the electrodes. The controller is programmed to operate in a monopolar mode and a bipolar mode. In the monopolar mode, the first and second electrodes are electrically uncoupled, and energy from the energy source is delivered to the first electrode in performing an ablation procedure. In the bipolar mode, first and second electrodes are electrically coupled and serve as opposite polarity poles to apply energy to a tissue target site, detect electrical signals at a tissue target site, or both.

### CROSS-REFERENCE TO RELATED APPLICATIONS

This application is a continuation-in-part of U.S. application Ser. No. 10/853,594, filed May 25, 2004, which is a continuation-in-part of U.S. application Ser. No. 10/056,807, filed Jan. 25, 2002, the entire teachings of both of which are incorporated herein by reference.

### BACKGROUND

The present disclosure relates to systems and methods for ablating and electrically interfacing with a patient's heart. More particularly, it relates to electrosurgical devices and related systems capable of performing ablation procedures as well as non-ablation procedures requiring electrical interface with cardiac tissue, such as mapping, stimulating, pacing, electrical signal monitoring/sensing, etc.

The heart includes a number of pathways that are responsible for the propagation of signals necessary to produce continuous, synchronized contractions. Each contraction cycle begins in the right atrium where a sinoatrial node initiates an electrical impulse. This impulse then spreads across the right atrium to the left atrium, stimulating the atria to contract. The chain reaction continues from the atria to the ventricles by passing through a pathway known as the atrioventricular (AV) node or junction, which acts as an electrical gateway to the ventricles. The AV junction delivers the signal to the ventricles while also slowing it, so the atria can relax before the ventricles contract.

Disturbances in the heart's electrical system may lead to various rhythmic problems that can cause the heart to beat irregularly, too fast or too slow. Irregular heart beats, or arrhythmia, are caused by physiological or pathological disturbances in the discharge of electrical impulses from the sinoatrial node, in the transmission of the signal through the heart tissue, or spontaneous, unexpected electrical signals generated within the heart. One type of arrhythmia is tachycardia, which is an abnormal rapidity of heart action. There are several different forms of atrial tachycardia, including atrial fibrillation and atrial flutter. With atrial fibrillation, instead of a single beat, numerous electrical impulses are generated by depolarizing tissue (thus creating depolarizing waves) at one or more locations in the atria (or possibly other locations). These unexpected electrical impulses produce irregular, often rapid heart beats in the atrial muscles and ventricles. Patient's experiencing atrial fibrillation may suffer from fatigue, activity intolerance, dizziness, and even strokes.

The precise cause of atrial fibrillation, and in particular the depolarizing tissue causing “extra” electrical signals, is currently unknown. As to the location of the depolarizing tissue, it is generally agreed that the undesired electrical impulses often originate in the left atrial region of the heart. Recent studies have expanded upon this general understanding, suggesting that nearly 90 percent of these “focal triggers” or electrical impulses are generated in one (or more) of the four pulmonary veins (PV) extending from the left atrium. In this regard, as the heart develops from an embryonic stage, left atrium tissue may grow or extend a short distance into one or more of the PVs. It has been postulated that this tissue may spontaneously depolarize, resulting an unexpected electrical impulse(s) propagating into the left atrium and along the various electrical pathways of the heart.

A variety of different atrial fibrillation treatment techniques are available including drugs, surgery, implants, and ablation. While drugs may be the treatment of choice for some patients, drugs typically only mask the symptoms and do not address the underlying cause. Implantable devices, on the other hand, usually correct an arrhythmia only after it occurs. Surgical and ablation treatments, in contrast, can actually cure the problem by removing and/or ablating the abnormal tissue or accessory pathway responsible for the atrial fibrillation (e.g., electrically isolating the abnormal tissue from the atrium pathway). Ablation treatments rely upon the application of destructive energy to the target tissue, including direct current electrical energy, radiofrequency electrical energy, laser energy, microwave energy, ultrasound energy, thermal energy, and the like. The energy applicator, such as an ablating electrode, is normally disposed along a distal portion of a catheter or other instrument. Ablation of the abnormal tissue or accessory pathway responsible for atrial fibrillation has proven highly viable.

As indicated above, for some treatments, the ablating electrode or other element can be formed or provided as part of a catheter that is delivered via the vascular system to the target site. While relatively non-invasive, catheter-based treatments present certain obstacles to achieving precisely located, complete ablation lesion patterns due to the highly flexible nature of the catheter itself, the confines of the target site, etc.

A highly viable alternative ablation device is a hand-held electrosurgical instrument. As used herein, the term “electrosurgical instrument” or “surgical instrument” includes a hand-held instrument capable of ablating tissue or cauterizing tissue, but does not include a catheter-based device. The electrosurgical instrument is relatively short (as compared to a catheter device), and rigidly couples the electrode (or a tip thereof) to the instrument's handle that is otherwise held and manipulated by the surgeon. The rigid construction of the electrosurgical instrument requires direct, open access to the targeted tissue. Thus, for treatment of atrial fibrillation via an electrosurgical instrument, it is desirable to gain access to the patient's heart through one or more openings in the patient's chest (such as via a sternotomy, a thoractomy, a small incision or port, etc.). In addition, the patient's heart may be opened through one or more incisions, thereby allowing access to the endocardial surface of the heart.

During use, once the target site (e.g., right atrium, left atrium, epicardial surface, endocardial surface, etc.) is accessible, the surgeon positions the electrode tip of the electrosurgical instrument at the target site. The electrode is then energized, ablating (or for some applications, cauterizing) the contacted tissue. A desired lesion pattern is then created (e.g., portions of a known “Maze” procedure) by moving the tip in a desired fashion along the target site. In this regard, the surgeon can easily control positioning and movement of the electrode tip, as the electrosurgical instrument is rigidly constructed and relatively short (in contrast to a catheter-based ablation technique).

Ablation of PV tissue may cause the PV to shrink or constrict due to the relatively small thickness of tissue formed within a PV. Because PVs have a relative small diameter, a stenosis may result from the ablation procedure. Even further, other vital bodily structures are directly adjacent each PV. These structures may be undesirably damaged when ablating within a PV. Therefore, a technique has been suggested whereby a continuous ablation lesion pattern is formed in the left atrium wall about the ostium associated with the PV in question. In other words, the PV is electrically isolated from the left atrium by forming an ablation lesion pattern that surrounds the PV ostium. As a result, any undesired electrical impulse generated within the PV will not propagate into the left atrium, thereby eliminating unexpected atria contraction.

Electrosurgical instruments, especially those used for the treatment of atrial fibrillation, have evolved to include additional features that provide improved results for particular procedures. For example, U.S. Pat. No. 5,897,553, the teachings of which are incorporated herein by reference, describes a fluid-assisted electrosurgical instrument that delivers a conductive solution to the target site in conjunction with the electrical energy, thereby creating a “virtual” electrode. The virtual electrode technique has proven highly effective in achieving desired ablation while minimizing collateral tissue damage. Other electrosurgical instrument advancements have likewise optimized system performance. For example, the Cardioblate® surgical instrument, available from Medtronic, Inc., incorporates a malleable shaft that allows the surgeon to shape (and re-shape) the shaft as desired to reach essentially any area of the heart, while also affording the surgeon an ability to handle the instrument in an ergonomically correct manner at all times. Similar features are described in U.S. application Ser. No. 10/056,807, filed Jan. 25, 2002, the teachings of which are incorporated herein by reference.

In connection with cardiac ablation procedures, surgeons often desire to identify the origination point of the undesired electrical impulses prior to ablation and/or to confirm that a formed ablation pattern has properly isolated and/or destroyed area(s) generating undesired electrical impulses. To this end, accepted techniques generally entail electrically interfacing with the patient's heart at one or more locations. For example, mapping may be accomplished by placing one or more mapping electrodes into contact with the tissue in question (e.g., the endocardial surface of the heart and/or the epicardial surface of the heart primarily within the region intended to have been isolated from the rest of the heart) and monitoring electrical signals propagating thereon (e.g., entrance block sensing). Also, the cardiac tissue can be stimulated using a pulsed current before and/or after an ablation procedure has been performed on a heart in order to determine how successful the ablation was (e.g., exit block pacing). Similarly, stimulating energy can be delivered to identify anatomical structure(s) of interest.

While the above-described ablation and related cardiac interface procedures can be performed using multiple instruments, surgeons prefer the simplicity of a single surgical instrument. For example, U.S. application Ser. No. 10/853,594 filed May 25, 2004 describes a surgical instrument for ablation and cardiac mapping using a monopolar energy source/electrode. U.S. Application Publication No. 2006/0161151 describes a surgical ablation and pacing device incorporating a removable tip. The tip is removed for ablation procedures, and attached to the instrument for pacing or sensing.

In light of the above, a need exists for electrosurgical instruments and related systems that readily facilitate multiple different electrical-based cardiac procedures, including tissue ablation and other non-ablation interactions.

### SUMMARY

Some aspects in accordance with principles of the present disclosure relate to a system for ablating tissue and electrically interfacing with a heart. The system includes a surgical instrument, an energy source, and a controller. The surgical instrument includes a shaft, a first electrode, a second electrode, and a non-conductive handle. The shaft defines a proximal section and a distal section, with the first and second electrodes being provided at the distal section. In this regard, the first and second electrodes are electrically insulated from one another at the distal section. Further, the proximal section of the shaft is coupled to the handle. The energy source is electrically connected to the surgical instrument, with the controller controlling delivery of energy from the energy source, as well as monitoring electrical signals from the electrodes. In this regard, the controller is programmed to operate in a monopolar mode and a bipolar mode. In the monopolar mode, the first and second electrodes are electrically uncoupled, and energy from the energy source is delivered to the first electrode in performing an ablation procedure. Conversely, in the bipolar mode, the first and second electrodes are electrically coupled and serve as opposite polarity poles. The opposite polarity poles serve to apply energy to a tissue target site, detect electrical signals at a tissue target site, or both, in the bipolar mode. For example, in some embodiments, the controller is programmed to perform a mapping or sensing procedure in the bipolar mode, and in other configurations to provide a pacing procedure in the bipolar mode. In yet other embodiments, the system further includes a grounding electrode apart from the surgical instrument, with the controller programmed to direct energy from the first electrode to the grounding electrode as part of an ablation procedure in the monopolar mode.

Yet other aspects in accordance with principles of the present disclosure relate to a surgical instrument for use in ablating tissue and electrically interfacing with a heart. The instrument includes a shaft, a first electrode, a second electrode, and a non-conductive handle. The shaft defines a proximal section and a distal section, with the first and second electrodes being provided at the distal section. In this regard, the electrodes are electrically insulated from one another at the distal section. Further, the first electrode differs from the second electrode in at least one of size, shape, or porosity. Finally, the non-conductive handle is coupled to the proximal section of the shaft. With this configuration, the surgical instrument is highly amenable for through-the-chest procedures on a patient's heart, including tissue ablation, as well as other procedures such as sensing, stimulating, pacing, etc. In some embodiments, the first electrode defines a rounded tip surface and is fluidly connected to a lumen provided with the shaft for distributing conductive fluid therefrom. In other configurations, the second electrode is a ring, with the first electrode positioned or extending distally from the second electrode in a co-axial fashion.

Yet other aspects in accordance with principles of the present disclosure relate to a method of treating a patient's heart. The method includes providing a surgical instrument having a shaft coupled to a handle and maintaining electrically-insulated first and second electrodes at a distal section thereof. The distal section is positioned through the chest of the patient in some embodiments, and a non-ablation procedure and an ablation procedure are performed. With the non-ablation procedure, the first and second electrodes are contacted against cardiac tissue, and are operated as opposite polarity poles. Further, the first and second electrodes are energized by way of either a separate energy source or by a depolarization wave propagating across the contacted cardiac tissue. Conversely, with the ablation procedure, the first electrode is contacted against the cardiac tissue, and is operated as a monopolar pole. Energy is delivered to the first electrode (and not the second electrode) from an energy source to create an ablation lesion pattern in the contacted tissue, thereby isolating an area of cardiac tissue. In some embodiments, the non-ablation procedure occurs prior to the ablation procedure; in other embodiments the ablation procedure occurs prior to the non-ablation procedure. Further, the non-ablation procedure can be a stimulating procedure in which a stimulating energy is passed between the first and second electrodes (e.g., in identifying a vagal nerve of the patient, pacing the patient's heart, etc.), and/or to sense a depolarization wave generated by the patient's heart.

### BRIEF DESCRIPTION OF THE DRAWINGS

FIG. 1 is a side view of an electrosurgical system in accordance with principles of the present disclosure, including portions shown in block form;

FIG. 2A is an enlarged, perspective view of a distal portion of an electrosurgical instrument useful with the system of FIG. 1;

FIG. 2B is an end view of the instrument of FIG. 2A;

FIG. 2C is an enlarged, cross-sectional view of a distal segment of the instrument of FIG. 2A;

FIGS. 3A and 3B generally illustrate possible use of the instrument of FIG. 2A in contacting tissue;

FIG. 4 is an exploded view of the instrument of FIG. 1;

FIG. 5 is an enlarged, perspective view of a portion of an insulative body component of the instrument of FIG. 4;

FIGS. 6A-6E illustrate assembly of the instrument of FIG. 4;

FIG. 7 is an enlarged, perspective, cross-sectional view of an alternative insulative body useful with the instrument of FIG. 4;

FIGS. 8A-8C are side views of the instrument of FIG. 1, illustrating exemplary shapes available during use of the instrument;

FIG. 9A is cut-away illustration of a patient's heart depicting use of an electrosurgical instrument in accordance with principles of the present disclosure in performing a surgical ablation procedure;

FIG. 9B is an enlarged illustration of a portion of FIG. 9A;

FIGS. 10A and 10B illustrate use of an electrosurgical system of FIG. 1 in performing non-ablation procedures in accordance with principles of the present disclosure;

FIGS. 11A and 11B illustrate use of the electrosurgical instrument in performing pacing and ablation procedures;

FIGS. 12A and 12B illustrate use of electrosurgical system of FIG. 1 in performing sensing and ablation procedures; and

FIGS. 13A and 13B are side views of a portion of an alternative electrosurgical instrument in accordance with principles of the present disclosure.

### DETAILED DESCRIPTION

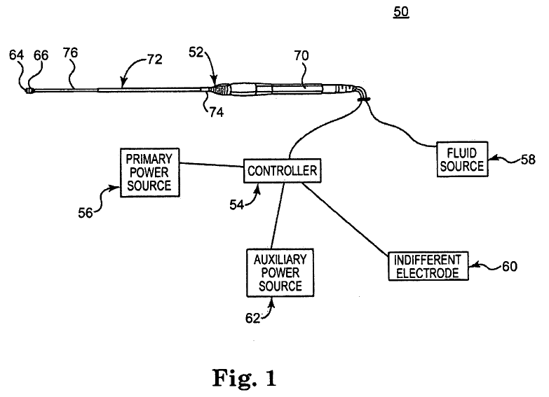

One configuration of an electrosurgical system 50 in accordance with aspects of the present disclosure is shown in FIG. 1. The system 50 includes an electrosurgical instrument 52, a controller 54, and a primary energy source 56. With some constructions described below, the surgical instrument 52 is configured to distribute conductive fluid to a target site, such that the system 50 can further include an optional fluid source 58. Further, in performing various procedures, the system 50 can optionally include an indifferent or grounding electrode 60 and/or an auxiliary or stimulating energy source 62. The various components are described in greater detail below. In general terms, however, the surgical instrument 52 is electronically connected to the controller 54 and in turn the primary source 56, with the surgical instrument 52 providing or carrying first and second electrodes 64, 66 (referenced generally). During use, the electrosurgical instrument 52 is deployed to position the first electrode 64, and in some instances the second electrode 66, into contact with targeted tissue. The controller 54 then operates to perform a desired procedure in which one or both of the electrodes 64, 66 electrically interface with cardiac tissue. For example, an ablation procedure can be performed whereby the controller 54 dictates delivery of an ablation energy from the energy source 56 to at least the first electrode 64. Where provided, conductive fluid can be delivered from the fluid source 58 to the surgical instrument 52 and subsequently distributed and energized at the target site in forming a virtual electrode that is capable of ablating or cauterizing contacted tissue. Where provided, the indifferent electrode 60 may also be employed in connection with the ablation procedure. With other, non-ablation procedures, the controller 54 effectuates delivery of a stimulating energy to one or both of the electrodes 64, 66 (e.g., via the optional auxiliary energy source 62) and/or monitors electrical signals generated at the electrodes 64, 66 by the contacted cardiac tissue.

### Electrosurgical Instrument

In addition to the first and second electrodes 64, 66, the electrosurgical instrument 52 includes a handle 70 and a shaft 72. The handle 70 and the shaft 72 can assume a variety of forms, but are generally constructed such that a proximal section 74 of the shaft 72 is coupled to the handle 70. Conversely, the first and second electrodes 64, 66 are provided at a distal section 76 of the shaft 72. In this regard, the shaft 72 includes or maintains various components effectuating electrical isolation of the first and second electrodes 64, 66 relative to one another, as well as conduction of electrical energy to, and electrical signals from, the electrodes 64, 66 and the controller 54 as described below.

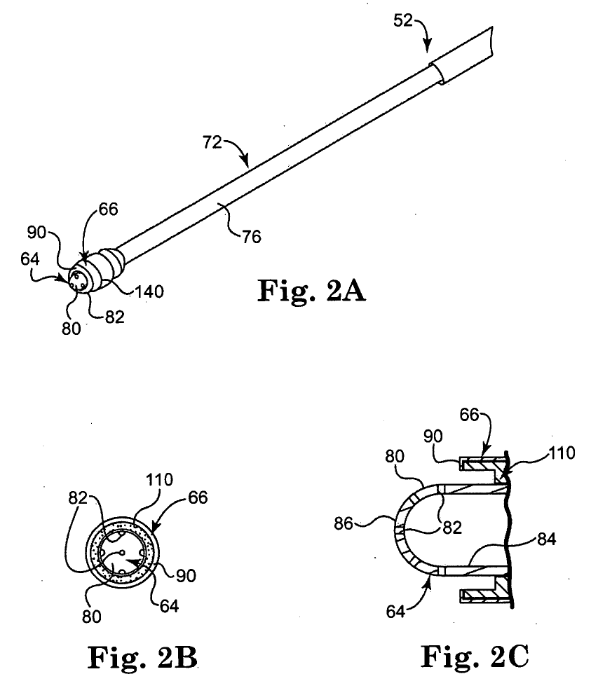

The electrodes 64, 66 can assume a variety of forms (e.g., made of an electrically conductive, surgically-safe material such as stainless steel, platinum-iridium, etc.), and are maintained in relatively close proximity to one another along the distal section 76. In this regard, the first and second electrodes 64, 66 can have differing constructions appropriate for performing a particular procedure. For example, the first electrode 64 can be configured for ablation procedures, with the second electrode 66 being configured for various non-ablation stimulation and/or sensing procedures (in combination with the first electrode 64 as described below). With this in mind, a construction of the first electrode 64 differs from a construction of the second electrode 66 in terms of at least one of size, shape, and/or porosity. For example, as shown in FIGS. 2A-2C, in some configurations the first electrode 64 has or defines a rounded tip surface 80 that is highly amenable to sliding movement across tissue as part of an ablation procedure. This rounded tip 80 configuration can have a uniform radius of curvature, and is thus indifferent to a rotational orientation of the electrosurgical instrument 52. That is to say, regardless of how a surgeon grasps the handle 70 (FIG. 1), a concentric profile of the electrode tip surface 80 in all directions (e.g., in front of the surgeon's thumb position, behind the surgeon's thumb position, etc.) is always the same so that the tip surface 80 is readily maneuvered along tissue (not shown) in any direction. Along these same lines, the first electrode 64 has, in some embodiments, a shape corresponding with a shape of the second electrode 66 (described below) such that upon final assembly, the first electrode 64 is concentric relative to the second electrode 66 (e.g., with some constructions in which the second electrode 66 has a ring-like construction, the first electrode 64 is shaped to be concentrically positioned within the second electrode 64). When making electrical measurements from the surface of the heart with this electrode pair 64, 66, the amplitude of measured signals is indifferent to a rotational orientation of the instrument 52. Alternatively, the tip surface 80 can have other shapes that may or may not be rounded and/or concentric.

In addition, with some constructions, the first electrode 64 forms a plurality of passages 82 that are fluidly connected to an internal lumen (not shown) provided with the shaft 72 as described below. As a point of reference, FIG. 2C generally shows a passageway 84 being formed by the first electrode 64. The passageway 84 can be fluidly connected to the lumen of the shaft 72, or the first electrode 64 can be integrally formed as part of an elongated shaft component such that the illustrated passageway 86 is part of the shaft lumen. One or more of the passages 82 can be formed at a distal-most end 84 of the first electrode 64 and/or proximal the distal-most end 86. Various arrangements of the passages 82 in accordance with principles of the present disclosure are described in U.S. application Ser. No. 10/056,807 filed Jan. 25, 2002. Alternatively, the first electrode 64 can be made of a porous material that allows fluid to pass from the internal lumen through the first electrode 64. As used herein, “porosity” is in reference to a structure that is inherently porous or that has passage(s) formed therethrough. In yet other embodiments, the first electrode 64 can be non-porous and the passages 82 eliminated (e.g., with applications in which a conductive fluid is not employed).

The second electrode 66 can also assume a variety of forms, but does not include any formed passages or is otherwise less porous as compared to a porosity of the first electrode 64 with some constructions. In some embodiments, the second electrode 66 is a ring or ring-like (e.g., an open-ended cup) and is coaxially disposed about the first electrode 64. Alternatively, the second electrode 66 can be formed to assume other shapes that may or may not be a ring (e.g., a partial ring). Regardless, the second electrode 66 forms or provides a distal face 90 that is exposed relative to the shaft 72, and thus available to contact and electrically interface with tissue. While the second electrode 66 is configured to deliver stimulation energy to contacted tissue, it is contemplated that the second electrode 66 will not be employed in performing an ablation procedure (e.g., delivering ablation energy) and can thus be less robust in construction as compared to the first electrode 64 and/or a surface area of the distal face 90 can be less than that of the tip surface 80 of the first electrode 64.

Upon final assembly, the first electrode 64 is spaced or offset from the second electrode 66 as best shown in FIG. 2C. With some constructions, the electrodes 64, 66 are laterally and longitudinally spaced from one another, with the first electrode 64 projecting distal the second electrode 66 (or the second electrode 66 being movable to a position proximal the first electrode 64). In this regard, a longitudinal distance between the distal-most end 86 of the first electrode 64 and a distal face 90 of the second electrode 66 is on the order of 0.1-10 mm, for example, on the order of 5 mm. As described below, this construction promotes placement of both the electrodes 64, 66 into contact with tissue with movement of the shaft 72. Further, a lateral spacing between the electrodes 64, 66 is on the order of 0.1-5 mm, for example, on the order of 1 mm.

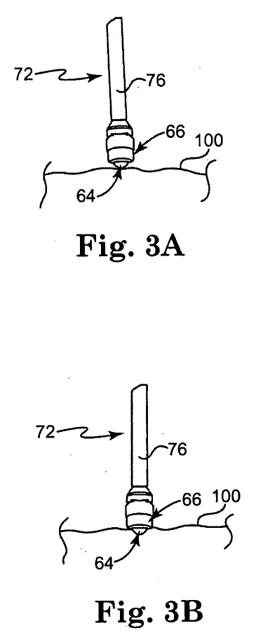

Application of the above-described arrangement of the electrodes 64, 66 in contacting tissue can generally be described with reference to FIGS. 3A and 3B. As shown in FIG. 3A, the first electrode 64 will initially contact a tissue surface 100 as the instrument 52, and in particular the distal section 76 of the shaft 72, is directed toward the tissue surface 100. By gently directing the shaft 72 distally from the position of FIG. 3A to the position of FIG. 3B, the second electrode 66 is also brought into contact with the tissue surface 100. As described below, with both electrodes 64, 66 in contact with the tissue surface 100, the electrodes 64, 66 can operate as opposite poles in sensing electrical activity along the tissue surface 100 and/or delivering a stimulation energy to the tissue surface 100. In this regard, the relatively small, fixed distance between the electrodes 64, 66 greatly reduces possible variability in the electrical pathway between the two components, reduces the risk of energy passing through the heart into inappropriate areas, and allows greater control over the amount of power required to prompt the intended response (especially as compared to conventional applications employing a grounding pad applied to the patient's body).

Returning to FIGS. 2A-2C, in addition to having different constructions, the first and second electrodes 64, 66 are electrically insulated or isolated from one another at the distal section 76 of the shaft 72 by an insulative body 110. The insulative body 110 can assume a variety of forms as described below (e.g., formed of an electrically non-conductive material), and generally encompasses a portion of an exterior of the first electrode 64 (and/or a conductor electrically coupled to the first electrode 64), with the second electrode 66 being assembled over the insulative body 110. Further, and as best shown in FIG. 2C, the insulative body 110 can dictate and maintain a desired lateral spacing between the electrodes 64, 66, as well as projection of the first electrode 64 distally beyond the second electrode 66 in some embodiments.

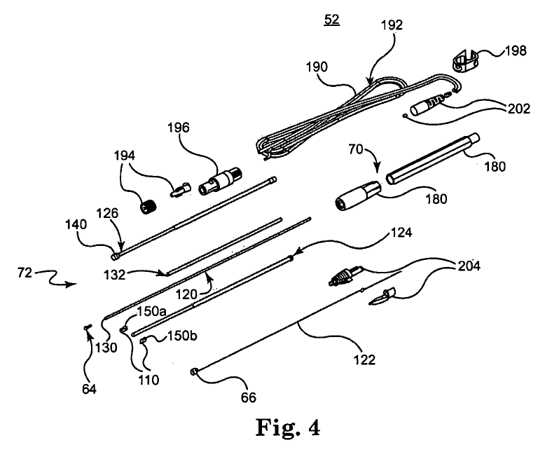

The first and second electrodes 64, 66 can be assembled to, and maintained by, the shaft 72 in a wide variety of manners, with the shaft 72 being constructed to facilitate operation of the electrodes 64, 66 as well as other desired features. For example, and as shown in FIG. 4, the shaft 72 can include a first conductor 120, a second conductor 122, an interior insulator 124, an exterior insulator 126, and the insulative body 110 (illustrated as an assembly in FIG. 4). In general terms, the first conductor 120 communicates electrical signals (and energy) to and from the first electrode 64, whereas the second conductor 122 communicates electrical signals (and/or energy) to and from the second electrode 66. The interior insulator 124 electrically isolates the first and second conductors 120, 122, whereas the exterior insulator 126 exteriorly insulates the second conductor 122. Finally, and as previously described, the insulative body 110 electrically isolates the first and second electrodes 64, 66.

The first conductor 120 is, in some configurations, an elongated tube formed of an electrically conductive material (e.g., stainless steel). With this configuration, the tubular first conductor 120 defines an internal lumen 130 (referenced generally) that is otherwise in fluid communication with the passages 82 (FIGS. 2A-2C) of the first electrode 64 as described above. The lumen 130 can extend through an entirety of the first conductor tube 120, or the first conductor tube 120 can include a solid segment, with fluid access to the lumen 130 being provided via one or more side ports. In yet other embodiments, the first conductor 120 is not tubular (e.g., is a solid body such as a wire) and thus does not form the lumen 130. In some configurations, the first conductor 120 is longitudinally reinforced by an optional support tube 132. The support tube 132 can be formed of an electrically conductive material (e.g., stainless steel), and has a length less than that of the first conductor tube 120 such that the support tube 132 does not extend to the distal section 76 (FIG. 1) of the shaft 72. In other embodiments, the support tube 132 is eliminated.

As described in greater detail below, the first conductor tube 120 as well as the support tube 132 (where provided) can be configured to impart a malleable or shapeable characteristic to the shaft 72. Thus, for example, the first conductor tube 120 and the support tube 132 are formed of a malleable material, such as fully annealed 304 stainless steel; however, other conductive materials such as, for example, Nitinol can be used. Regardless, the first conductor tube 120 and the first electrode 64 combine, upon final assembly, to define an elongated electrode body, with the passages 82 (FIGS. 2A-2C) in the first electrode 64 being fluidly connected to the internal lumen 130. Alternatively, the first electrode 64 and the first conductor tube 120 can be integrally formed. Further, and/or in addition, the first conductor tube 120 can include or form one or more of the passages 82 (apart from the first electrode 64).

Connection between the first conductor tube 120 and the first electrode 64 can be accomplished in a variety of manners. For example, the first conductor tube 120 can be connected to the first electrode 64 via an acceptable coupling technique including, for example, welding, laser welding, spin welding, crimping, gluing, soldering, and press-fitting. Alternatively, a distal end of the first conductor tube 120 and the first electrode 64 can be configured for threadable engagement with one another and/or mechanical engagement member(s) (e.g., pins, screws, rivets, etc.) can be employed. In some constructions, the first electrode 64 is rigidly coupled to the first conductor tube 120. In other configurations, the first electrode 64 is movably coupled to the first conductor tube 120, whereby the first electrode 64 can be moved and/or locked relative to the first conductor tube 120.

The second conductor 122 is connected to, and extends proximally from, the second electrode 66 and in some embodiments is a wire. As described below, the second electrode 66 is used in performing various low power-type procedures (e.g., non-ablation procedures) such that the second conductor wire 122 can be a low power wire. Alternatively, the second conductor 122 can assume a variety of other forms appropriate for communicating electrical signals/energy to and from the second electrode 66.

The interior insulator 124 encompasses a substantial portion of the first conductor 120, electrically insulating the first conductor 120 from the second conductor 122. With this in mind, the interior insulator 124 can be a sheath or sleeve formed of one or more electrically non-conductive materials (e.g., a silicone sleeve). In some alternative constructions, multiple layers of electrically non-conductive materials are employed that assist in preventing the likelihood of forming an electrical short along the length of the interior insulator 120 due to a mechanical failure of one of the non-conductive materials. In this regard, the first conductive sheath 124 is, with some configurations, comprised of two materials having considerably different mechanical properties (e.g., a silicone and a fluoropolymer). For example, a silicone tubing material can be overlayed with a heat shrink fluoropolymer tubing material. Alternatively, the interior insulator 124 can be one or more non-conductive coatings applied over a portion of the first conductor tube 120 (as well as the support tube 132 where provided). In addition to being non-conductive, the interior insulation 124 is preferably flexible and conforms to the first conductor 120 (as well as the support tube 132 where provided) such that the interior insulator 124 does not impede desired shaping and re-shaping of the shaft 72 as described below. The interior insulator 124 can assume a variety of other forms apart from the sleeve or sheath construction illustrated in FIG. 4 and capable of ensuring electrical isolation between the first and second conductors 120, 122. In fact, where the second conductor 122 is sufficiently self-insulated (e.g., a wire over-molded with non-conductive material), the interior insulator 124 can be eliminated (with the first conductor being externally insulated by the exterior insulator 126).

The exterior insulator 126 is, with some embodiments, similar in construction to the interior insulator 124 described above (e.g., a sheath or sleeve). In general terms, then, the exterior insulator 124 is formed of one or more electrically non-conductive materials (e.g., polyvinylchloride), and serves to electrically insulate the encompassed portion of the second conductor 122 (as well as enhance exterior insulation of the first conductor 120) upon final assembly. As shown in FIG. 2A, the exterior insulator 126 is configured to conform to the shape(s) of the components being encompassed therein. Thus, for example, a distal end 140 of the exterior insulator 126 can form an enlarged diameter, commensurate with an outer diameter of the second electrode 66 as described below. As shown, the distal end 140 terminates proximal the distal face 90 of the second electrode 66 to allow electrical interaction with the second electrode 66. Alternatively, and returning to FIG. 4, the exterior insulator 126 can assume a variety of other forms, and need not necessarily encompass the first conductor 120. Rather, the exterior insulator 126 serves to enhance a visual appearance of the shaft, and ensure that the second conductor wire 122 and a portion of the second electrode 66 are externally insulated along the shaft 72. In other embodiments, the exterior insulator 126 can be eliminated.

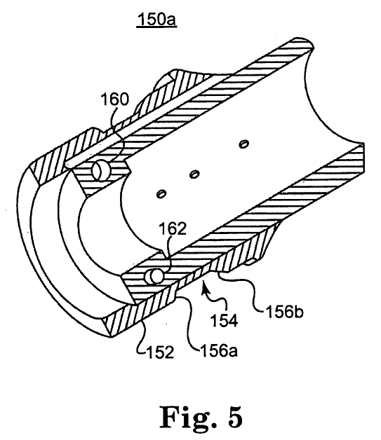

As a point of reference, the first conductor tube 120 and the first electrode 64 can be akin to an electrosurgical instrument available from Medtronic, Inc., under the trade name Cardioblate®. With this but one acceptable construction, the second electrode 66 and the second conductor wire 122 are configured to be fitted to the existing instrument design. Thus, the insulative body 110 is, in some configurations, constructed for assembly about the first conductor tube 120 and to receive the second electrode 66 in a manner ensuring that the second electrode 66 is electrically isolated from the first electrode 64 as well as the first conductor tube 120. For example, the insulative body 110 can include first and second halves 150a, 150b that combine to define the insulative body 110 as an annular component upon final assembly. With additional reference to FIG. 5, each of the insulative body halves 150a, 150b has an outer surface 152 defining a capture zone 154. The capture zone 154 can be provided in a variety of fashions, and generally includes opposing shoulders 156a, 156b sized to receive a corresponding feature of the second electrode 66. In addition, each insulative body half 150a, 150b includes or forms one or more features that facilitate assembly of the components 150a, 150b, for example an aperture 160 and a protrusion 162. The aperture 160 is sized to frictionally receive and maintain the protrusion 162 such that the protrusion 162 of the first half 150a nests within the aperture 160 of the second half 150b, and vice-versa. A wide variety of other constructions can be employed with the insulative body halves 150a, 150b to facilitate assembly to the shaft 72. Other features associated with some embodiments of the insulative body 110 are described below. It will be understood, however, that the insulative body 110 can assume a number of other forms that may or may not include a plurality of components assembled to one another.

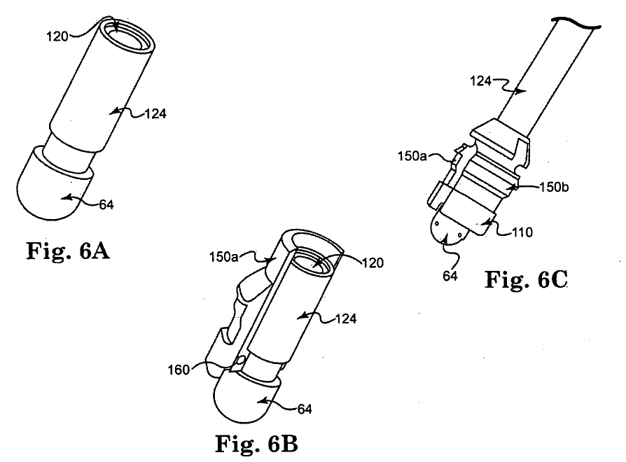

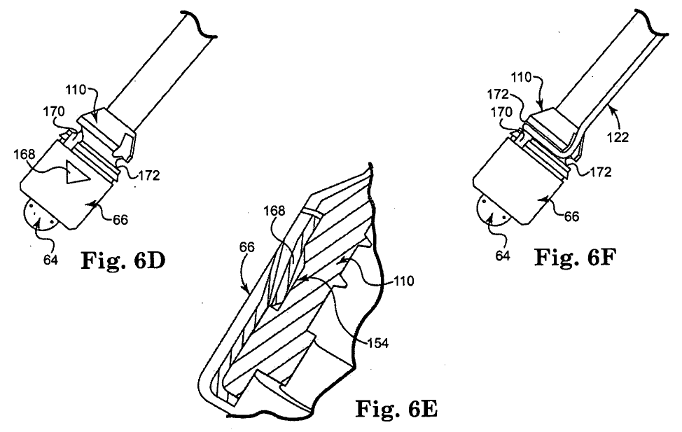

With the but one acceptable construction of the insulative body 110 described above, assembly of the shaft 72 can include forming or affixing the first electrode 64 to the first conductor tube 120. The interior insulator 124 is then applied over the first conductor tube 120 (as well as the support tube 132 where provided). A portion of the resultant assembly is shown in FIG. 6A. The insulative body 110 is then assembled to the first conductor tube 120/first electrode 64 by placing the first half 150a about the first conductor tube 120/interior insulator 124 as shown in FIG. 6B. The second half 150b (FIG. 4) is similarly assembled to the conductor tube 120/interior insulator 124, with the assembly features 160, 162 (FIG. 5) securing the halves 150a, 150b to one another in a collet-style arrangement. As shown in FIG. 6C, then, the halves 150a, 150b combine to define the completed insulative body 110, with the capture zones 154 associated with each of the halves 150a, 150b being aligned with one another.

The second electrode 66 is then assembled to the insulative body 110 as shown in FIG. 6D. For example, where the second electrode 66 has a ring-type construction, the second electrode 66 is coaxially disposed over the insulative body 110, with the first electrode 64 extending distally therefrom. As shown in FIG. 6E, the second electrode 66 is affixed to the insulative body 110, for example via one or more barbs 168 engaged with the capture zone 154 (as a point of reference, the first electrode 64, the first conductor tube 120, and the interior insulator 124 are omitted from the view of FIG. 6E). A wide variety of other techniques can be employed for affixing the second electrode 66 to the insulative body 110. Regardless, the insulative body 110 electrically isolates the second electrode 66 from the first electrode 64 (FIG. 6D). Further, the insulative body 110 ensures and maintains the desired spacing between the electrodes 64, 66 as previously described with respect to FIG. 2C.

It will be understood that the above description of the insulative body 110 as including the halves 150a, 150b (FIG. 4) is but one acceptable construction in accordance with principles of the present disclosure. For example, the insulative body 110 can be over-molded or adhered to the first conductor 120/interior insulator 124. Regardless, following assembly of the second electrode 66 to the insulative body 110, the second conductor wire 122 is positioned, for example as shown in FIG. 6F. The second conductor wire 122 can be affixed to the second electrode 66 (e.g., welded) prior to assembly over the insulative body 110. With this but one acceptable construction, the second conductor wire 122 is extended proximally from the second electrode 66, for example along a longitudinal slot 170 defined by the insulative body 110. Where desired, the second conductor wire 122 is circumferentially wrapped about at least a portion of the insulative body 110 to provide strain relief. In this regard, the insulative body 110 can further form a circumferential groove 172 sized to receive the second conductor wire 122. By routing the second conductor wire 122 around the insulative body 110, any force that is applied to the second conductor wire 122 is redirected from a shear force to a tightening force.

Once assembled, the second conductor wire 122 is extended along an exterior of the interior insulator 124. Returning to FIG. 4, the exterior insulator 126 is then applied over the wire 122 and the insulative body 110 such that at least a portion of the second electrode 66 is exteriorly exposed (as shown in FIG. 2A).

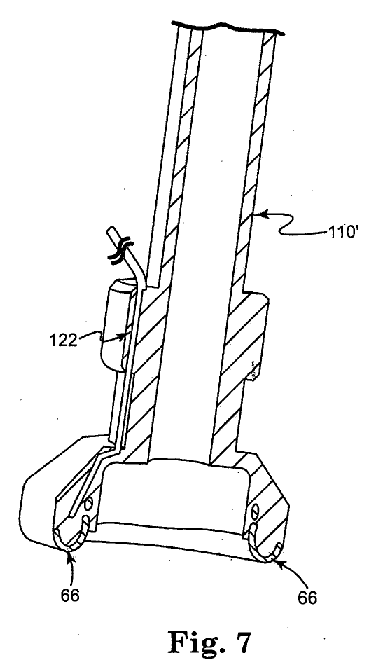

A wide variety of other techniques and components can alternatively be employed for assembling the second electrode 66 to the shaft 72 in a manner that electrically isolates the second electrode 66 from the first electrode 64 and the first conductor 120. For example, the second electrode 66 can be movably maintained relative to the shaft 72 in a manner that allows the second electrode 66 to articulate to match a presented angle of the first electrode 64 relative to tissue being contacted. With this in mind, FIG. 7 illustrates an alternative embodiment insulative body 110′ formed of a flexible, non-conductive material (e.g., a flexible polymer) over-molded to the second electrode 66. The wire 122 can also be over-molded into the insulative body 110′ or otherwise assembled thereto so as to establish an electrical connection to the second electrode 66. Regardless, the insulative body 110/second electrode 66 is assembled to the first conductor 120/first electrode 64 (FIG. 6A) as previously described. With this construction, the flexible interface body 110′ allows the second electrode 66 to pivot independent of the first electrode 64. In fact, with some alternative constructions, a separate mechanism (not shown) can be deployed to “flip” the second electrode 66 proximally away from the first electrode 64, for example prior to an ablation procedure. In other, related embodiments, the insulative body 110′ is sized to extend an entire length of the first conductor 120, thus serving as the interior insulator 124. Even further, the wire 122 can extend entirely within the elongated insulative body 110′, thereby eliminating a need for a separate exterior insulator.

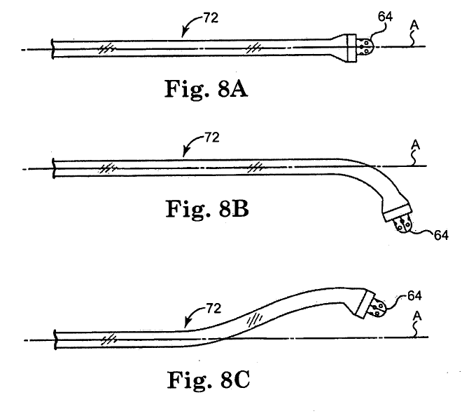

Regardless of an exact construction of the insulative body 110, the resultant shaft 72 (FIG. 2A) is, in some embodiments, malleable or shapeable. With reference to FIGS. 8A-8C, the shaft 72 is configured to be transitionable from an initial straight state (FIG. 8A) to a bent or curved state (FIGS. 8B and 8C). In this regard, the electrosurgical instrument 52, and in particular the shaft 72, is initially presented to a surgeon in the straight state of FIG. 8A, whereby the shaft 72 assumes a straight shape defining a central axis A. In the straight state, the shaft 72 is indifferent to rotational orientation, such that the electrosurgical instrument 52 (FIG. 1) can be grasped at any rotational position and the electrodes 64, 66 will be located at an identical spatial position. Subsequently, depending upon the constraints of a particular electrosurgical procedure, the shaft 72 can be bent relative to the central axis A. Two examples of an applicable bent state or shape are provided in FIGS. 8B and 8C. In some embodiments, the shaft 72 can be bent at any point along a length thereof, and can be formed to include multiple bends or curves. Regardless, the shaft 72 is configured to independently maintain the shape associated with a selected bent shape. That is to say, the shaft 72 does not require additional components (e.g., pull wires, etc.) to maintain the selected bent shape. Further, the shaft 72 is constructed such that a user can readily re-shape the shaft 72 back to the straight state of FIG. 8A and/or other desired bent configurations. Notably, the shaft 72 is configured to relatively rigidly maintain the selected shape such that when a sliding force is imparted onto the shaft 72 as the first electrode 64 is dragged across the tissue, the shaft 72 will not overtly deflect from the selected shape. Alternatively, the shaft 72 can be highly rigid and not configured to be shaped by a surgeon.

Other components of the electrosurgical instrument 52 in accordance with some embodiments are also shown in FIG. 4. For example, the handle 70 can include one or more sections 180 that effectuate rigid connection to the shaft 72. Regardless, the handle 70 is made of a sterilizable, rigid, and non-conductive material, such as a polymer or ceramic. Further, the handle 70 is, in some embodiments, ergonomically designed to comfortably rest within a surgeon's hand, and has a circular shape in cross-section. This configuration facilitates grasping of a handle 70, and thus of the electrosurgical instrument 52, at any position along the handle 70 regardless of an overall rotational orientation of the electrosurgical instrument 72.

Other optional features associated with the electrosurgical instrument 52 include a power cable 190 and fluid tubing 192. The power cable 190 carries separate wirings that are electrically coupled within the handle 70 to the first and second conductors 120, 122, respectively. The fluid tubing 192 is fluidly connected to the internal lumen 130 of the first conductor tube 120. With this arrangement, then, the power cable 190 electrically couples the conductors 120, 122 to one or more separate energy sources (e.g., the primary energy source 56 and the auxiliary energy source 62 of FIG. 1), whereas the fluid tubing 192 fluidly connects the internal lumen 130 with a separate fluid source (e.g., the fluid source 58 of FIG. 1). Other optional components, such as a Luer lock assembly 194, a pin connector 196, a pinch clamp 198, a strain collar assembly 202, and a transition over-mold assembly 204 are also shown in FIG. 4.

It should be understood that the above descriptions of the electrosurgical instrument 52 are but a few configurations envisioned by the present disclosure. In general terms, any instrument configuration in which two electrodes (e.g., the first and second electrodes 64, 66) are provided, with at least one of the electrodes (and related conductor) being appropriate for a tissue ablation procedure, are acceptable. Thus, while the electrosurgical instrument 52 has been described as operating to generate a virtual electrode via the delivery of an electrically conductive fluid, in other embodiments conventional, conductive fluid is not used.

### Controller

Returning to FIG. 1, the controller 54 includes a computer or other logic circuitry capable of effectuating one or more of the procedures described below (e.g., hardware or software programs). While the controller 54 and the primary power source 56 are illustrated in FIG. 1 as separate components, in some embodiments, the primary power source 56 is provided with the controller 54 (e.g., akin to a Cardioblate® 68000 Generator available from Medtronic, Inc., of Minneapolis, Minn.). Further, the controller 54 can be programmed to effectuate performance of only some of the procedures described below (e.g., ablation procedures), and a second controller (not shown) maintaining appropriate programming employed to perform others of the procedures (e.g., one or more non-ablation procedures). The controller 54 can be adapted to establish an electronic link with the second controller (e.g., a Model 2090/2290 Programmer/Analyzer and/or a Model 5388/5348 External Temporary Pacemaker, all available from Medtronic, Inc.). As used throughout this specification, then, reference to a “controller” includes a single controller or two or more electronically linked controllers or computing devices.

The controller 54 is programmed to operate the electrosurgical instrument 52 in a monopolar mode and a bipolar mode as described in greater detail below. In general terms, in the monopolar mode, the controller 54 operates to deliver energy from the primary energy source 56 only to the first electrode 64 (and thus not to the second electrode 66). For example, the controller 54 operates to deliver energy only to the first conductor 120 (FIG. 4) while maintaining an internal electrical isolation relative to the second conductor 122 (FIG. 4), and thus the second electrode 66. In other words, relative to operation of the controller 54 in the monopolar mode, the first and second electrodes 64, 66 are electrically uncoupled. Conversely, in the bipolar mode, the controller 54 operates the first and second electrodes 64, 66 as oppositely charged poles, effectively establishing an electrical coupling between the first and second electrodes 64, 66. In the bipolar mode, then, energy can be passed between the first and second electrodes 64, 66, or an electrical signal passing between the first and second electrodes 64, 66 can be sensed.

### Ablation Procedures

The electrosurgical system 50 can perform a cardiac ablation procedure in the monopolar mode. For example, the electrosurgical system 50 can be employed for the surgical treatment of cardiac arrhythmia, and in particular treatment of atrial fibrillation via ablation of the atrial tissue. To this end, the Maze procedure, such as described in Cardiovascular Device Update, Vol. 1, No. 4, July 1995, pp. 2-3, the teachings of which are incorporated herein by reference, is a well-known technique whereby lesion patterns are created along specified areas of the atria. The Maze III procedure, a modified version of the original Maze procedure, has been described in Cardiac Surgery Operative Technique, Mosby Inc., 1997, pp. 410-419, the teachings of which are incorporated herein by reference. In an effort to reduce the complexity of the surgical Maze procedure, a modified Maze procedure was developed and described The Surgical Treatment of Atrial Fibrillation, Medtronic, Inc., 2001, the teachings of which are incorporated herein by reference.

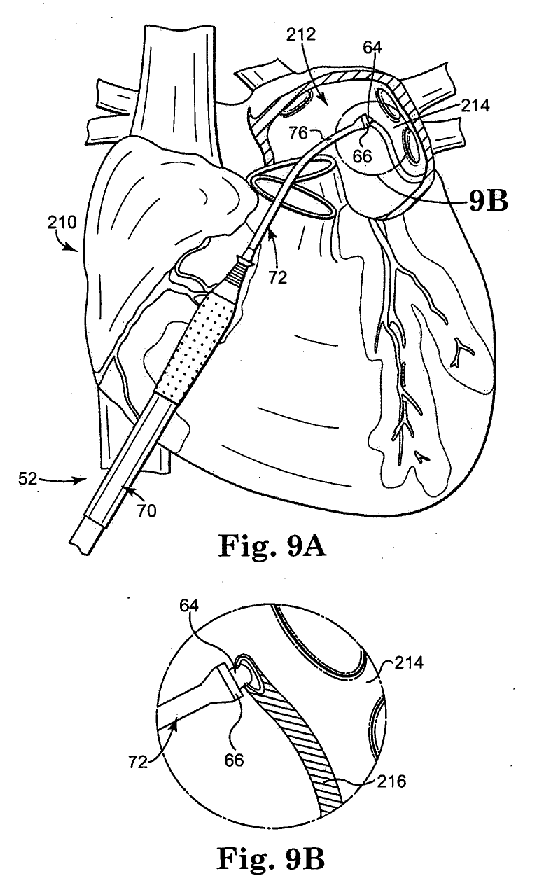

With the above in mind, one procedure that can be performed using the electrosurgical system 50 is a cardiac ablation procedure, such as all or a portion of the Maze procedure(s) described above. To this end, FIG. 9A includes a representation of a heart 210 with its left atrium 212 exposed. Prior to use, the indifferent electrode 60 (FIG. 1), such as a grounding electrode pad, needle electrode, or grounding wire attached to a metal element (e.g., metal retractor), is secured to the patient's body (e.g., the patient's back). With applications in which the electrosurgical instrument 52 is configured to establish or create a virtual electrode via delivery of a conductive fluid, the fluid source 58 (FIG. 1) is fluidly connected to the instrument 52. Similarly, where the shaft 72 is provided with a malleable construction, the shaft 72 is initially provided to the surgeon in the straight state (FIG. 8A). The surgeon then evaluates the constraints presented by a tissue target site 214 and the desired lesion pattern to be formed. Following this evaluation, the surgeon determines an optimal shape of the shaft 72 most conducive to achieving the desired ablation/lesion pattern. With this evaluation in mind, the surgeon then transitions or bends the shaft 72 from the initial straight state to the bent state illustrated in FIG. 9A. With other embodiments, the shaft 72 is not independently bendable and thus is provided to the surgeon in a shape that cannot be changed.

Regardless of whether the shaft 72 permits selection of a desired shape, the distal section 76 is directed toward the tissue target site 214. Conductive fluid from the fluid source 58 (FIG. 1) is delivered to the tissue target site 214 via the internal lumen 130 (FIG. 4), the passages 82 (FIGS. 2A-2C), and/or a porous nature of the first electrode 64. Prior to or following initiation of fluid flow, the distal section 76 is maneuvered to place the first electrode 64 into contact with tissue of the target site 214. Once sufficient fluid flow has been established, the controller 54 (FIG. 1) operates to energize the first electrode 64 with an ablation energy via the primary energy source 56 (FIG. 1). In this regard, the controller 54 operates the electrosurgical instrument 52 as a monopolar device, whereby energy is delivered only to the first electrode 64 (passing through the patient's body to the indifferent electrode 60). The second electrode 66 may or may not be in contact with tissue of the target site 214; regardless, energy does not flow from the first electrode 64 to the second electrode 66. That is to say, the electrodes 64, 66 (and corresponding conductors 120, 122 (FIG. 4)) are electrically uncoupled. The first electrode 64 energizes the distributed fluid, thereby creating a virtual electrode that ablates contacted tissue (or, the energized first electrode 64 ablates contacted tissue where the electrosurgical instrument 52 does not employ delivery of conductive liquid). The surgeon then slides or drags the first electrode 64 along the left atrium 212 tissue, thereby creating a desired lesion pattern 216, as best shown in FIG. 9B. The rigid coupling between the shaft 72 and the handle 70 allows the first electrode 64 to easily be slid along the atrial tissue via movement of the handle 70. Once the desired lesion pattern 216 has been completed, energization of the first electrode 64 is discontinued, as well as delivery of conductive fluid from the fluid source 58 (where provided). If additional lesion patterns are required, the surgeon can again evaluate the selected target tissue site and, with some embodiments, re-shape the shaft 72 accordingly.

With cardiac ablation procedures in accordance with some aspects of the present disclosure, radio frequency energy is employed, with the electrosurgical instrument 50 (and the corresponding controller 54 and the primary power source 56) adapted to deliver a maximum of 30 watts of power at 500 KHz for two minutes. Other ablation parameters (e.g., energy type, voltage, current, frequency, etc.) can alternatively be employed.

The target site 214 shown in FIG. 9A is but one acceptable location of a cardiac ablation procedure in accordance with the present disclosure. Virtually any tissue surface of a human heart can be accessed and ablated using the electrosurgical system 50. Further, the electrosurgical instrument 52 can be delivered to the target site 214 in a wide variety of manners, such as open chest, through a thoractomy, through a sternotomy, percutaneously, transveneously, endoscopically, for example through a percutaneous port, through a stab wound or puncture, through a small incision, for example in the chest or in the abdomen, or in combinations thereof. Additionally, access to the target site 214 can be gained via open-chest surgery on a heart in which the sternum is split and the rib cage opened with a retractor.

### Non-Ablation Procedures

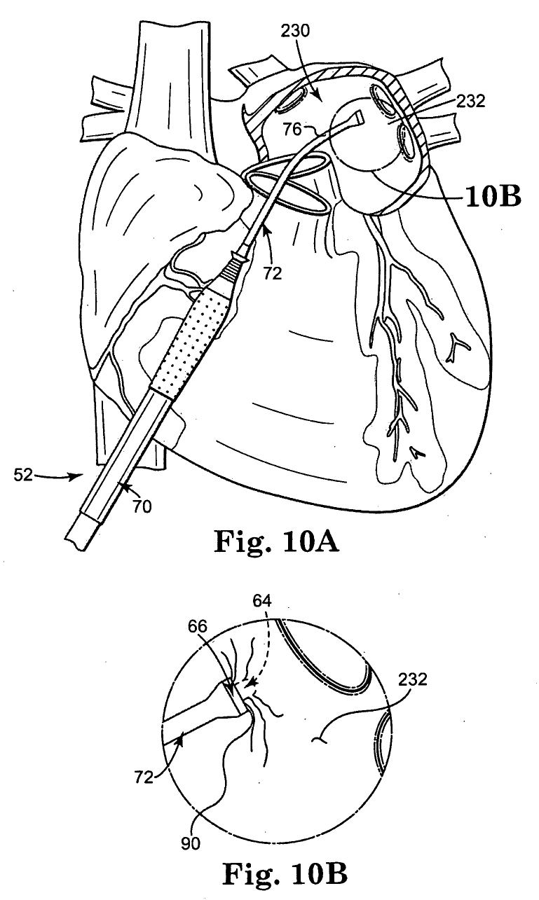

In addition to the ablation procedure described above in which the controller 54 (FIG. 1) operates the electrosurgical instrument 52 in a monopolar mode, the electrosurgical system 50 can further be used to perform a number of other procedures in which the electrosurgical instrument 52 electrically interfaces with cardiac tissue and with the controller 54 operating in a bipolar mode. In general terms, the bipolar mode of operation entails the distal section 76 being delivered to a tissue target site 230 as shown in FIGS. 10A and 10B via one or more of the techniques described above. The electrosurgical instrument 52 is manipulated such that the first electrode 64 contacts a tissue surface 232 of the target site 230 in a manner generally commensurate part of the delivery of the first electrode 64 as with the ablation procedure described above. Unlike the ablation procedure, however, the electrosurgical instrument 52 is additionally manipulated to gently depress the distal section 76 further toward the tissue surface 232 such that at least a portion of the distal face 90 of the second electrode 66 also contacts the tissue surface 232 (with the tissue surface 232 generally conforming to a shape of the first electrode 64 as in FIG. 10B). In this regard, an entirety of the distal face 90 of the second electrode 66 can be in contact with the tissue surface 232 as shown; alternatively, the shaft 72 can be shaped or oriented (e.g., angularly offset relative to a plane of the tissue surface 232) such that only a portion of the distal face 90 is in contact with the tissue surface 232. Regardless, the first and second electrodes 64, 66 are both in contact with the tissue surface 232, and thus positioned to effectuate electrical interaction with the tissue surface 232, with the controller 54 operating the first and second electrodes 64, 66 as opposite polarity poles in connection with the bipolar mode of operation. Several non-ablation procedures available in the bipolar mode are described below.

For example, the controller 54 (FIG. 1) can be programmed to effectuate performance of a non-ablation, stimulation procedure in which a stimulating energy is delivered to the tissue surface 232 in the bipolar mode. With some approaches in accordance with principles of the present disclosure, the stimulating energy is delivered and passed between the first and second electrodes 64, 66 (with the first electrode 64 serving as a negative pole and the second electrode 66 serving as a positive pole, or vice-versa) to assist in identifying an anatomical structure such as terminations of the sympathetic and parasympathetic nervous systems around the heart. In this regard, the controller 54 operates to deliver stimulating energy, in a controlled fashion, from the primary power source 56 (FIG. 1) and/or the auxiliary energy source 62 (FIG. 1) at an output that is selected by the user or pre-programmed to the controller 54. For example, different frequency and/or components of current or voltage are selected or set at an appropriate level for a specific procedure in stimulating nerve(s) to be identified. Stimulation parameters, in some embodiments, include a rate in the range of 5 Hz-50 Hz, a pulse width in the range of 0.1 msec-10.0 msec, and an amplitude of 1-10 volts. Alternatively, other stimulation signal parameters are also envisioned.

In general terms, when the stimulating energy is administered in close proximity to a nerve structure of interest (e.g., ganglionated plexus, vagal nerve, etc.), an involuntary or autonomic patient response will occur. The patient's heart rate may drop and/or a significant and rapid drop in the patient's blood pressure may occur. With this in mind, one bipolar mode stimulation procedure entails the surgeon positioning the first and second electrodes 64, 66 against the tissue surface 232 at an approximate, expected location of a nerve structure or other anatomical structure of interest. The stimulating energy is then delivered and the patient monitored for possible response. For example, the patient's heart rate can be monitored. Alternatively, an ECG system used in conjunction with the electrosurgical system 50 can be programmed to issue a warning or other indication (audible or visual) when the heart rate drops by a certain percentage. Similarly, the patient's blood pressure can be monitored. Alternatively, an ECG system can be programmed to give an indication when the patient's blood pressure rapidly drops by a certain percentage. Where the applied stimulation energy does not produce a vagal response, the surgeon repositions the distal section 76 such that the first and second electrodes 64, 66 contact a different area of the target tissue site 230. The process is repeated (i.e., stimulating energy applied and patient response monitored) until the anatomical structure in question is located.

An additional and/or alternative bipolar mode, non-ablation procedure in accordance with aspects of the present disclosure is a pacing procedure. In general terms, the heart is “paced” by a low frequency signal from an external energy source to control the beating rate of the heart. Typically, a beating rate of 20 to 30 beats per minute faster than the patient's then-current heart rate is chosen. When the heart rate is controlled by the external energy source, the pacing is considered to have “captured” control of the heart.

The pacing procedure entails the distal section 76 of the shaft 72 being directed toward the target site 230 such that the first and second electrodes 64, 66 are both in contact with the tissue surface 232 as described above. The controller 54 (FIG. 1) then operates to deliver stimulating or pacing energy to the electrodes 64, 66 (with the first electrode 64 operating as a positive pole and the second electrode 66 acting as a negative pole, or vice-versa). Effectively, then, the electrodes 64, 66 are electrically coupled so that energy passes between the electrodes 64, 66. In this regard, the controller 54 can deliver energy from the primary energy source 56. Alternatively, the pacing energy can be generated by the auxiliary energy source 62 (FIG. 1). For example, the auxiliary energy source 62 can be an external temporary pacemaker, such as an External Temporary Pacemaker available from Medtronic, Inc., under Model 5348 or Model 5388. As alluded to above, the energy source 62 can further include a separate controller programmed to effectuate performance of the pacing procedure. With this construction, the external temporary pacemaker 62 is electronically linked to the controller 54 and serves to control delivery of an energy from a contained energy source to the surgical instrument 52 via the controller 54.

With some configurations, a pacing threshold for the electrosurgical instrument 52 for pacing atrial tissue is less than 10 mA at 0.5 msec using Medtronic's Model 5388 Pacemaker. Medtronic's Model 5388 External Temporary Pacemaker has a maximum output of 20 mA. In the context of use on heart tissue, if the heart does not respond to an initial pulsed current, the current may be increased until the heart rate responds to the stimulation. The stimulation or pacing energy can be increased or decreased to attain capture where desired. For example, a pacing amplitude in the range of 0.1-10.0 volts and a current in the range of 0.1-25 mA can be provided.

Yet another non-ablation procedure available in some embodiments with the controller 54 (FIG. 1) operating in the bipolar mode is a sensing procedure in which electrical activity propagating along cardiac tissue is monitored or sensed. Once again, the first and second electrodes 64, 66 are placed into contact with the tissue surface 232 as described above. The controller 54 effectively establishes an electrical coupling between the first and second electrodes 64, 66, for example by operating the first electrode 64 as a positive pole and the second electrode 66 as a negative pole (or vice-versa). In contrast to previous applications, however, the controller 54 does not delivery energy to electrodes 64 or 66. Instead, an electrical signal (typically a voltage measurement) progressing across the electrodes 64, 66 is monitored. For example, intrinsic electrical activity across the contacted tissue 232 (e.g., a depolarizing wave) will progress from the first electrode 64 to the second electrode 66 (or vice-versa). As the depolarizing wave progresses from the first electrode 64 to the second electrode 66 (or vice-versa), the controller 54 (or an electronically-linked analyzer) monitors or senses the changing electrical signal(s), and can record or otherwise note various attributes.

With configurations in which the first electrode 64 is concentrically arranged relative to the second electrode 66, the amplitude of the measured signal is insensitive to a rotational orientation of the electrode pair 64, 66. It should be understood, however, that a measurement taken from a pair of discrete electrode tips separated by some distance would produce differing amplitude measured signals depending upon the rotational orientation of the electrode pair in relation to the conduction vector of the electrical depolarization wavefront passing directionally through the heart muscle. The signal amplitude (e.g., voltage) would be highest when the electrodes were in-line with the conduction vector. When the electrodes were transverse to the wavefront direction, the signal (e.g., voltage) would be at minimum amplitude. Regardless, in some embodiments, the system 50 is adapted to display a sensed EGM at a display gain rate of 1 mv-10 mv, with a maximum signal impedance of 4000Ω (e.g., impedance in the range of 200Ω-4000Ω) at a frequency of 25-50 Hz.

### Combined Procedures

One or more of the above-described, non-ablation procedures can be used by a surgeon to identify a proper location of a subsequently performed ablation therapy and/or evaluate efficacy of a previously-formed ablation lesion pattern. For example, the stimulating procedure can be used to identify a desired anatomical structure (e.g., via vagal response) as described above. Once the anatomical structure of interest has been identified, the electrosurgical system 50 can then be operated to form an ablation lesion pattern along a location indicated by the identified anatomical structure. For example, endocardial ablation at or near identified ganglionated plexi can eliminate the vagal response to stimulation and high-frequency fractionated potentials. Notably, while the surgeon may desire to clean the distal section 76 of the shaft 72 following the stimulation procedure and prior to the ablation procedure, no other re-configuration of the electrosurgical instrument 52 is required. That is to say, the first electrode 64, as otherwise used for the ablation procedure, is also used with the stimulation procedure (along with the second electrode 66), such that the surgeon need not remove the distal section 76 from the target site 230. Thus, the electrosurgical instrument 52 is employed, without modification, in performing the non-ablation and ablation procedures. Where desired, following ablation, the stimulation procedure can be repeated to gauge an effectiveness of the ablative therapy. For example, a target site that was expected to have been electrically isolated by the ablation lesion pattern can be subjected to the stimulating energy as described above; where the stimulating energy does not cause the vagal response, isolation can be confirmed. Once again, the electrosurgical instrument 52 need not be modified, and can remain in close proximity to the target site 230, when transitioning from the ablation procedure to the stimulating procedure (and vice-versa)

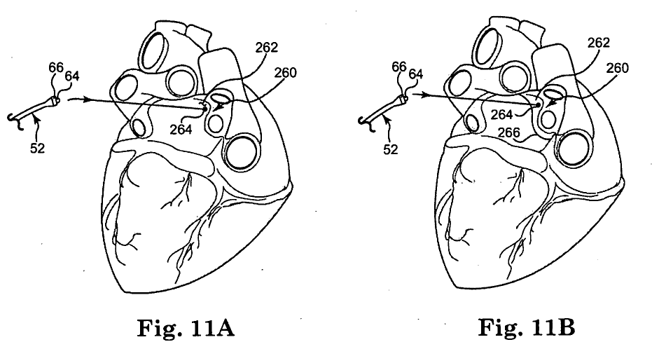

Similarly, the pacing procedure described above can be performed before and/or after the ablation procedure. As a point of reference, the pacing procedure can be utilized in the context of exit block sensing of the heart. Prior to ablation the distal section 76 is placed at a target tissue site 250/260 to be electrically isolated as shown in FIG. 11A. With the first and second electrodes 64, 66 both in contact with tissue 262 of the target site 260 (point 264 in FIG. 11A), the controller 54 (FIG. 1) either alone or as dictated by the auxiliary power source 62 (FIG. 1), operates the electrodes 64, 66 in the bipolar mode and delivers pacing energy sufficient to effectuate “capture” of the heart. Various parameters associated with this capture (e.g., power, beats per minute, etc.) can then be recorded via the controller 54 (or the auxiliary power source 62).

The pacing procedure is then discontinued, and the electrosurgical instrument 52 operated in the monopolar mode to perform an ablation procedure as described above to form a lesion pattern. One exemplary lesion pattern 266 is shown in FIG. 11B. The electrosurgical instrument 52 need not be re-configured or modified in transitioning from the pacing procedure to the ablation procedure; in fact, in some embodiments, the distal section 76 remains at the target site 260. Once the ablation pattern 264/isolated area has been created, it can then be tested for electrical isolation by repeating the pacing procedure described above.

In particular, the first and second electrodes 64, 66 are again placed in contact with the tissue 262 of the target site 260, within the area to be tested for conduction and pacing capture (e.g., approximately at the point 264). The controller 54 (FIG. 1), either alone or as dictated by the auxiliary energy source 62 and associated controller then operates the first and second electrodes 64, 66 in a bipolar mode to perform a pacing procedure (i.e., delivering the pacing energy), attempting to “capture” the patient's heart as described above. With some approaches, the previously-recorded power settings are initially employed, and a determination is made as to whether the heart is “captured” at these same settings. Where the heart cannot be captured using the same pre-ablation power settings, an initial determination can be made that the ablation lesion pattern 266 was successful in isolating the target site 260. In other embodiments, if capture is not achieved at the pre-ablation power settings, the power output can then be increased (e.g., doubled) and a determination made as to whether the heart is “captured” at this increased power output. If capture is not achieved at this double power heart pacing, the area can be considered to be isolated and exit blocking from this area proven. Conversely, where the heart is captured during the post-ablation pacing procedure, an indication is given that the ablation lesion pattern 266 was not successful in isolating the target site, and the surgeon can then repeat the ablation procedure and/or form additional lesion pattern(s) at other areas.

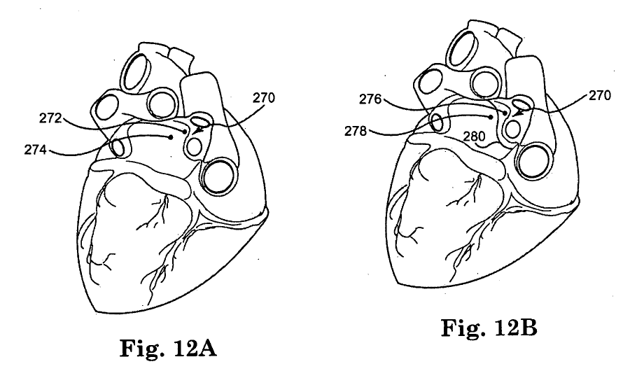

The sensing procedure can also be used to evaluate the effectiveness of an ablative therapy. As point of reference, the sensing procedure can be used in conjunction with entrance block testing of the heart. For example, FIG. 12A references at 270 an area of a heart to be isolated from extraneous electrical signals. With this in mind and with additional reference to FIG. 1, the distal section 76 is manipulated to position the first and second electrodes 64, 66 in contact with tissue at a first point 272 within the area to be isolated 270 and at a second point 274 outside of the area to be isolated 270. At each of the locations 272, 274, the electrical activity (e.g., sensed de-polarizing wave) is monitored and recorded via the controller 54. Other characteristics can also be determined or noted, such as difference (of any) in electrical activity between the points 272, 274.

The electrosurgical system 50 is then operated in the monopolar mode to perform an ablation procedure as described above. Once again, the electrosurgical instrument 52 need not be re-configured in transitioning from the sensing procedure to the ablation procedure.

Once the ablation therapy is complete, the electrosurgical system 50 is operated to repeat (in the bipolar mode) the sensing procedure described above. For example, and as shown in FIG. 12B, third and fourth locations 276 and 278 within, and outside of, the area to be isolated 270 are monitored for electrical activity, and various characteristics noted. As a point of reference, the third point 276 generally corresponds with a first point 272 (FIG. 12A), and the fourth point 278 generally corresponds with the second point 274 (FIG. 12A). Successful ablative isolation of the area 270 can be demonstrated, for example, by significant drop in the electrical activity across the “isolated” portion of the heart following formation of the ablation lesion 280 (e.g., difference in sensed electrical activity between the third and fourth points 276, 278) as compared to the electrical activity prior to ablation (e.g., difference in sensed electrical activity between the first and second points 272, 274).

In other embodiments, the initial monitoring steps (e.g., monitoring electrical activity prior to ablation) can be omitted as the surgeon becomes comfortable with the technology. In other words, the electrical activity monitoring can only be performed following ablation at points within and outside of the area to be isolated 270 (e.g., comparing electrical activity sensed at the third and fourth points 276, 278). Regardless, the monitoring output may be recorded and saved as a visual “ECG” type output and the collection of monitored information visually compared to each other. Alternatively, an algorithm can be programmed to the controller 54 and used to compare the captured outputs; if the different between the isolated area output (the third point 276) and the other outputs (the points 272, 274, and/or 278) are significant (or insignificant), then an audible or visual indication can be given.

While the above non-ablation procedures have been described in the context of using the electrosurgical system 50 to perform an ablation procedure (before and/or after the ablation procedure), in other embodiments, the non-ablation procedure can be performed alone. That is to say, methods in accordance with the principles of the present disclosure include the electrosurgical system 50 not being employed to directly perform an ablation procedure. In some embodiments, for example, the electrosurgical system 50 can be used in combination with a separate ablation device, providing concurrent sensing information useful in evaluating an efficacy of lesion(s) formed by the separate ablation device. For example, the electrosurgical system 50 can be operated to perform a sensing procedure (e.g., the first and second electrodes 64, 66 placed in contact with desired tissue), and electrical activity across the electrodes 64, 66 sensed and monitored. With the instrument 52 maintained in this tissue contact position (and the sensed signal being monitored/recorded), the separate ablation/isolation device is positioned and activated. For example, a bipolar clamping instrument is but one example of an available separate ablation device (e.g., a BP2® ablation clamp instrument available from Medtronic, Inc.). With this approach, as the isolation instrument is placed and clamped down, a reduction in the size of the sensed/recorded signal via the electrosurgical system 50 should be noted as the electrical pathway is narrowed/restricted. The separate isolation instrument can then be activated to create its isolating effect. Again, as this is performed, the surgeon will be able to detect, real-time, the effect of the attempted isolation by reviewing the output sensed by the electrosurgical system 50. In general terms, where the sensed electrical activity drops to nearly zero, efficacy of the applied lesion pattern can be confirmed.

Alternatively, the separate isolation instrument can be delivered and operated to form a lesion pattern. Subsequently, the electrosurgical system 50 can be operated to determine whether electrical isolation has occurred (e.g., the sensing procedure described above). Similarly, the pacing/exit blocking procedures described above can be employed (via the electrosurgical system 50) in evaluating the efficacy of an isolation procedure performed by a separate system. Other procedures requiring electrical interface with the heart, such as mapping and lead placement as described in U.S. application Ser. No. 10/854,594, can also be performed.

As made clear by the above, the electrosurgical system 50 is highly useful in performing a number of different procedures requiring electrical interface with cardiac tissue, including ablation and non-ablation procedures. With some constructions, “transitioning” of the electrosurgical system 50 from the monopolar mode to the bipolar mode (and vice-versa) can be effectuated by the surgeon using various auxiliary components, such as a footswitch. In other constructions, however, appropriate components are provided as part of the electrosurgical instrument 52 to itself. Under these circumstances, it may be desirable to provide two or more buttons or other actuators on the electrosurgical instrument 52 to facilitate selection of a desired procedure by the surgeon. To this end, certain procedures will require the delivery of energy to one or both of the electrodes 64 and/or 66 (e.g., ablation procedures or stimulation procedures), while others do not require delivery of energy (e.g., sensing procedures). With this in mind, in some embodiments, the electrosurgical instrument 52 is provided with features that ergonomically present the various actuators to the surgeon for interface, while obscuring or otherwise shielding one or more other actuator buttons not otherwise implicated by the particular procedure being performed.

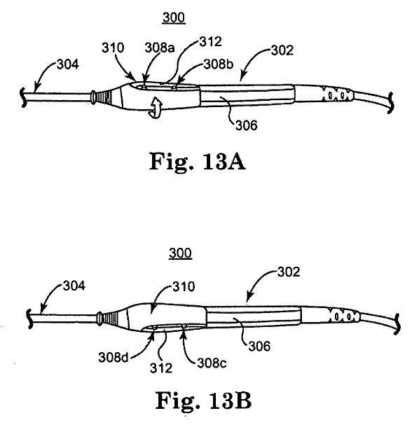

For example, FIG. 13A illustrates a portion of an alternative electrosurgical instrument 300 in accordance with principles of the present disclosure. The instrument 300 includes a handle 302 and a shaft 304. The shaft 304 is coupled to the handle 302, and incorporates first and second electrodes (not shown) at a distal section thereof as previously described. The handle is akin to the handle 70 (FIG. 1) previously described, and generally includes a handle body 306. In addition, the handle 302 include a plurality of actuators 308 and a shield body 310. The actuators 308 are electronically connected to the controller 54 (FIG. 1) via wiring (not shown) carried within the handle body 306. Further, the handle body 306 carries or maintains the actuators 308 so as to be exteriorly accessible by the user. The shield body 310 is movably coupled to the handle body 306 and selectively “exposes” one or more of the actuators 308 as described below.

The actuators 308 can assume a wide variety of forms, and in some embodiments are buttons or switches. Regardless, each of the actuators 308 are dedicated to initiate performance of a particular operation. With this in mind, the actuators 308 can be organized along the handle body 306 such that corresponding or similar dedicated operations are presented as a set of the actuators 308. By way of example only, FIG. 13A illustrates two the actuators 308a and 308b, whereas FIG. 13B illustrates two additional actuators 308c and 308d. It will be understood that any other number of the actuators 308 (either greater or lesser) is also acceptable. With this but one example, the first actuator 308a can be associated with an ablation procedure (e.g., actuation of the first actuator 308a initiates delivery of ablative energy to the first electrode 64 (FIG. 1) in a monopolar mode and delivery of conductive liquid (where provided)). The second actuator 308b relates to a stimulating procedure in which stimulating energy is delivered to the electrodes 64, 66 (FIG. 1) in a bipolar mode, but at a level less than that associated with the ablation energy. The third actuator 308c relates to a pacing procedure described above, such that actuation of the third actuator 308c initiates delivery of a pacing energy to the electrodes 64, 66. Finally, the fourth actuator 308d relates to a sensing operation, prompting the controller 54 to freeze or record a particular, sensed signal.

With the above constraints in mind, the shield body 310 is movable relative to the handle body 306 to selectively expose (and selectively cover) one or more of the actuators 308. For example, the shield body 310 can be rotatably coupled to the handle body 306, and forms one or more slots 312 sized to exteriorly expose one or more of the actuators 308. As a point of reference, in the rotational state of FIG. 13A, the shield body 310 is positioned such that the slot 312 is aligned with the first and second actuators 308a, 308b. Thus, a user can directly interface with either of the first or second actuators 308a or 308b. Conversely, the third and fourth actuators 308c, 308d (FIG. 13B) are encompassed by the shield body 310 and thus cannot be inadvertently actuated (e.g., depressed) by the user. In the rotational state of FIG. 13B, the third and fourth actuators 308c, 308d are exposed, and the first and second actuators 308a, 308b (FIG. 13A) are covered.

As a point of reference, the shield body 310 need not necessarily be rotationally mounted to the handle body 306. Instead, the shield body 310 can be slidably attached to the handle body 306 or coupled via any other construction/mechanism capable of covering one or more of the actuators 308 while revealing or uncovering one or more other of the actuators 308. Regardless, this configuration “clears” the electrosurgical instrument 300 of excess actuators 308 that are not being used for a particular procedure. Leaving unnecessary actuators 308 exposed may increase the risk of accidental activation and may otherwise compromise the ergonomics and control of the electrosurgical instrument 300.

### EXAMPLES AND COMPARATIVE EXAMPLES

The following examples and comparative examples further describe electrosurgical instruments and systems in accordance with the present disclosure, and testing performed to confirm various attributes of the electrosurgical instruments and systems. The examples are provided for exemplary purposes to facilitate an understanding of the present disclosure, and should not be construed to limit the disclosure to the examples.

Various tests were performed to evaluate the performance of electrosurgical instruments in accordance with the present disclosure in performing various procedures, and in particular electrical interface with tissue at various rotational positions of the bipolar device relative to the tissue that might otherwise affect the measured depolarization (electrogram) amplitude. An example electrosurgical instrument of the present disclosure was prepared in accordance with FIGS. 2A-2C (“Example-MAPS”). A first comparative example electrosurgical device (“Comparative A”) was prepared by assembling two surgical pacing and mapping tools available under the trade name Detect® Surgical Pacing and Mapping Tool, from Medtronic, Inc., of Minneapolis, Minn. (Model No. 10650) were assembled to one another, with the corresponding electrode surfaces having a center-to-center distance of 6.23 mm. A second comparative example electrosurgical instrument (“Comparative B”) was created by assembling one of the Detect® devices above to a surgical ablation pen device available from Medtronic, Inc., under the trade designation Cardioblate® XL Surgical Ablation Pen (Model No. 60814). The resultant electrosurgical instrument had a center-to-center electrode distance of 6.57 mm. A third comparative example electrosurgical instrument (“Comparative C”) was provided as a commercially available electrosurgical pen using dry RF energy.

The example electrosurgical instrument and the three comparative example electrosurgical instruments were each connected to a programmer/analyzer module available from Medtronic, Inc., under the trade designation CareLink® (Model No. 2090/2290). Each of the electrosurgical instruments were then employed to perform epicardial bipolar EGM sensing at the right pulmonary vein-atrial junctions and ventricle of animal test subjects. In particular, the electrodes of the electrosurgical device were placed into contact with cardiac tissue, and the resultant electrical signals (if any) sensed by the electrosurgical instrument recorded (as displayed on the programmer). Once initial sensing was achieved, measurements (peak-peak) were collected while rotating the devices clockwise, with the average values obtained at various rotational positions of the electrosurgical instrument recorded in Table I (atria) and Table II (ventricle) below.

TABLE I
Atrial EGM Measurements

| Device | Peak-Peak (millivolts) | Angle (°) |
|---|---|---|
| Example MAPS | 3.8 | Baseline |
| | 4.2 | 0 |
| | 3.3 | 45 |
| | 4.5 | 90 |
| | 4.6 | 180 |
| Comparative A | 2.1 | Baseline |
| | 2.4 | 0 |
| | 3.7 | 45 |
| | 4.1 | 90 |
| | 4.0 | 180 |
| Comparative B | 6.0 | Baseline |
| | 4.5 | 0 |
| | 5.0 | 45 |
| | 4.3 | 90 |
| | 4.2 | 180 |
| Comparative C | 2.8 | Baseline |
| | 3.5 | 0 |
| | 8.0 | 45 |
| | 4.5 | 90 |
| | 4.2 | 180 |

TABLE II
Ventricular EGM Measurements

| Device | Peak-Peak (millivolts) | Angle (°) |
|---|---|---|
| Example MAPS | 10.1 | 0 |
| | 10.1 | 45 |
| | 10.1 | 90 |
| | 10.1 | 180 |
| Comparative C | 5.1 | 0 |
| | 18.9 | 90 |

As evidenced by Tables I and II above, the electrosurgical instruments in accordance with aspects of the present invention are surprisingly found to provide a more consistent sensed amplitude as compared to the comparative example devices, irrespective of a rotational position of the corresponding electrodes relative to the tissue being monitored.

Although the present disclosure has been described with reference to preferred embodiments, workers skilled in the art will recognize that changes can be made in form and detail without departing from the spirit and scope of the present disclosure. For example, devices incorporating one or more of the features described herein can be employed in interfacing with cardiac tissue in a variety of manners, including open chest (e.g., stemotomy), minimally invasive (e.g., thoractomy, sub-xyphoid), or closed chest (e.g., port endoscopic) procedures.

What is claimed is:

1. A system for ablating tissue and electrically interfacing with a heart, the system comprising:
an electrosurgical instrument including:
a shaft defining a proximal section and a distal section,
a first electrode provided at the distal section,
a second electrode provided at the distal section and electrically isolated from the first electrode,
a non-conductive handle coupled to the proximal section of the shaft;
a first energy source electrically connected to the electrosurgical instrument; and
a controller controlling delivery of energy from the energy source and monitoring electrical signals from the electrodes, the controller programmed to operate in:
a monopolar mode in which the first and second electrodes are electrically uncoupled and energy from the energy source is delivered to the first electrode in performing an ablation procedure,
a bipolar mode in which the electrodes are electrically coupled and serve as opposite polarity poles in at least one of applying energy to, and detecting electrical signals at, a tissue target site in performing a non-ablation procedure.

2. The system of claim 1, wherein the controller is programmed to perform a sensing procedure upon electrical signals at the first and second electrodes in the bipolar mode.

3. The system of claim 1, wherein the controller is programmed to perform a stimulating procedure in delivering energy between the first and second electrodes in the bipolar mode.

4. The system of claim 1, wherein the controller is programmed to effectuate delivery of ablation energy to the first electrode in effectuating an ablation procedure and a pacing energy to the first and second electrodes in effectuating a non-ablation pacing procedure.

5. The system of claim 4, further comprising:
a second energy source connected to the controller;
wherein the controller is programmed to deliver the ablation energy from the first energy source in effectuating the ablation procedure and to deliver the pacing energy from the second energy source in effectuating the pacing procedure.

6. The system of claim 1, wherein the first and second electrodes are permanently affixed to the shaft.

7. The system of claim 1, wherein the first electrode has a characteristic different from the second electrode selected from the group consisting of size, shape, and porosity.

8. The system of claim 7, wherein the first electrode is more porous than the second electrode.

9. The system of claim 1, wherein the first electrode is concentrically arranged relative to the second electrode.

10. The system of claim 9, wherein detection of electrical signals at the electrodes in the bipolar mode is indifferent to a rotational orientation of the instrument.

11. The system of claim 1, wherein the first electrode defines a rounded tip surface.

12. The system of claim 11, wherein the shaft forms a lumen and the first electrode forms at least one passage fluidly connected to the lumen for distributing fluid from the lumen outwardly from the shaft, the system further comprising:
a source of conductive fluid fluidly connected to the lumen.

13. The system of claim 12, wherein the second electrode forms a distal face that is spatially offset from the rounded tip surface.

14. The system of claim 13, wherein the rounded tip surface is distal the distal face.

15. The system of claim 13, wherein the second electrode is a ring.

16. The system of claim 15, wherein the first electrode is co-axially positioned relative to the second electrode.

17. The system of claim 15, wherein the second electrode is coupled to a connector body that is movably assembled to the shaft.

18. The system of claim 1, wherein the shaft includes:
a first conductor in electrical communication with the first electrode; and
a second conductor in electrical communication with the second electrode;
wherein the first and second conductors are electrically isolated from one another.

19. The system of claim 18, wherein the first conductor is an electrically conductive tube.

20. The system of claim 19, wherein the shaft further includes:
an interior insulator disposed between the conductive tube and the second conductor; and
an exterior insulator exteriorly surrounding the second conductor and the first insulative body.

21. The system of claim 1, wherein the handle includes:
a handle body;
a first actuator maintained by the handle body and electronically connected to the controller for indicating initiation of an ablation procedure; and
a second actuator maintained by the handle body and electronically connected to the controller for indicating initiation of a stimulation procedure.

22. The system of claim 21, wherein the handle further includes:
a shield body movably assembled to the handle body and configured to selectively uncover only one of the first and second actuators.

23. The system of claim 1, wherein the instrument is adapted to interface with cardiac tissue through a chest of the patient.

24. The system of claim 1, further comprising:
a grounding electrode electrically connected to the controller;
wherein the monopolar mode includes conducting energy from the first electrode to the grounding electrode.

25. An electrosurgical instrument for use in ablating tissue and electrically interfacing with a heart, the instrument comprising:
a shaft defining a proximal section and a distal section;
a first electrode provided at the distal section;
a second electrode provided at the distal section and electrically insulated from the first electrode;
wherein the first electrode has a characteristic different from the second electrode selected from the group consisting of size, shape, and porosity; and
a non-conductive handle coupled to the proximal section of the shaft.

26. The instrument of claim 25, wherein the first electrode is more porous than the second electrode.

27. The instrument of claim 25, wherein the first electrode is concentrically arranged relative to the second electrode.

28. The instrument of claim 25, wherein the first electrode defines a rounded tip surface.

29. The instrument of claim 28, wherein the shaft forms a lumen and the first electrode forms at least one passage fluidly connected to the lumen for distributing fluid from the lumen outwardly from the shaft.

30. The instrument of claim 28, wherein the second electrode forms a distal face that is spatially offset from the rounded tip surface.

31. The instrument of claim 30, wherein the rounded tip surface is distal the distal face.

32. The instrument of claim 30, wherein the second electrode is a ring.

33. The instrument of claim 32, wherein the second electrode is coupled to a connector body that is movably assembled to the shaft.

34. The instrument of claim 25, wherein the shaft includes:
a first conductor in electrical communication with the first electrode; and
a second conductor in electrical communication with the second electrode;
wherein the first and second conductors are electrically isolated from one another.

35. The instrument of claim 34, wherein the first conductor is an electrically conductive tube.

36. The instrument of claim 35, wherein the shaft further includes:
an interior insulator body disposed between the conductive tube and the second conductor; and
an exterior insulator exteriorly surrounding the second conductor and the first insulative body.

37. The instrument of claim 25, wherein the handle includes:
a handle body;
a first actuator maintained by the handle body for indicating initiation of an ablation procedure; and
a second shield maintained by the handle body for indicating initiation of a stimulation procedure.

38. The instrument of claim 37, wherein the handle further includes:
a shield body movably assembled to the handle body and configured to selectively uncover only one of the first and second actuators.

39. The instrument of claim 25, wherein the instrument is adapted to interface with cardiac tissue through a chest of the patient.

40. A method for treating a patient's heart, the method comprising:
providing a surgical instrument including:
a shaft defining a proximal section and a distal section,
a first electrode provided at the distal section,
a second electrode provided at the distal section and electrically isolated from the first electrode,
a non-conductive handle coupled to the proximal section of the shaft;
performing a non-ablation procedure including:
contacting the first and second electrodes against cardiac tissue,
operating the first and second electrode as opposite polarity poles,
energizing the first and the second electrodes by at least one of an energy from an energy source and a depolarization wave propagating across the contacted cardiac tissue; and
performing an ablation procedure including:
contacting the first electrode against the cardiac tissue,
operating the first electrode as a monopolar pole,
delivering energy to only the first electrode from an energy source to create an ablation lesion to isolate an area of cardiac tissue.

41. The method of claim 40, wherein the non-ablation procedure occurs prior to the ablation procedure.

42. The method of claim 40, wherein the non-ablation procedure occurs after the ablation procedure.

43. The method of claim 40, wherein the non-ablation and ablation procedures are both performed at a target site and further include delivering the distal section to the target site, and further wherein the method includes the distal section remaining proximate the target site in transitioning between the non-ablation and ablation procedures

44. The method of claim 40, wherein the instrument further comprises a lumen within the shaft and in fluid communication with at least one passage formed in the first electrode, and further wherein performing an ablation procedure includes:
dispensing conductive fluid from the lumen via the at least one passage while delivering energy to the first electrode.

45. The method of claim 44, wherein the non-ablation procedure is characterized by the absence of dispensing conductive fluid from the lumen.

46. The method of claim 40, wherein the non-ablation procedure includes sensing an electrical measurement of the cardiac tissue via the second electrodes, and further wherein the sensed electrical measurement is indifferent to rotational orientation of the instrument.

47. The method of claim 40, wherein the non-ablation procedure includes:
passing a stimulating energy between the first and second electrodes while the first and second electrodes are in contact with the cardiac tissue; and
evaluating a response of the patient to the applied stimulating energy.

48. The method of claim 47, wherein the response is selected from the group consisting of heart rate and blood pressure.

49. The method of claim 48, wherein the non-ablation procedure further includes:
determining whether a location of the electrodes on the cardiac tissue is proximate a nerve of the patient based upon the evaluated response.

50. The method of claim 40, wherein the non-ablation procedure includes:
passing a pacing energy between the first and second electrodes while the electrodes are in contact with the cardiac tissue.

51. The method of claim 40, wherein the non-ablation procedure includes:
sensing a depolarization wave generated by the patient's heart at the first and second electrodes.

52. The method of claim 40, wherein a characteristic of the first electrode differs from the second electrode, the characteristic selected from the group consisting of size, shape, and porosity.

53. The method of claim 52, wherein the first electrode is concentrically arranged relative to the second electrode.

54. The method of claim 40, wherein performing an ablation procedure includes:
applying a grounding electrode to a body of the patient;
wherein the energy delivered to the first electrode passes to the grounding electrode.

55. The method of claim 40, wherein:
the handle includes:
a handle body,
a first actuator maintained by the handle body,
a second actuator maintained by the handle body;
the non-ablation procedure includes actuating the first actuator; and
the ablation procedure includes actuating the second actuator.

56. The method of claim 55, wherein the handle further includes a shield body movably assembled to the handle body, and further wherein performing the non-ablation procedure includes:
moving the shield body to cover the second actuator and uncover the first actuator.
